(19) United States
(12) Patent Application Publication
(10) Pub. No.: US 2025/0194973 A1
(43) Pub. Date: Jun. 19, 2025

(54) SINGLE USE LANCING DEVICE

(71) Applicant: Owen Mumford Limited, Woodstock, Oxfordshire (GB)

(72) Inventor: Matthew David FARMER, Woodstock, Oxfordshire (GB)

(21) Appl. No.: 18/849,247

(22) PCT Filed: Mar. 27, 2023
(86) PCT No.: PCT/EP2023/057771

§ 371 Date: Sep. 20, 2024

(30) Foreign Application Priority Data
Mar. 28, 2022 (GB) 2204315.2

(51) Int. Cl. A61B 5/15; A61B 5/151

(52) U.S. Cl. CPC A61B 5/150656; A61B 5/150022; A61B 5/15029; A61B 5/150389; A61B 5/150412; A61B 5/150595; A61B 5/150709; A61B 5/150916; A61B 5/15144

(57) ABSTRACT

A lancing device comprising a housing and a lancet. The lancet comprises a needle and is configured to be movable within the housing from an operational position to a retracted position. The lancing device further comprises a shroud coupled movably to the housing and initially covering the needle, and a biasing member coupled between the lancet and the shroud. The shroud is configured to, when pressed against a sampling site, move rearwards relative to the housing and, at a first point of the rearward movement, allow the needle to contact the sampling site and subsequently, at a second point of the rearward movement, release energy stored in the biasing member to drive the lancet from the operational position to the retracted position. The housing comprises lancet retaining features configured to retain the lancet in the operational position until the shroud reaches the second point of the rearward movement.

### TECHNICAL FIELD

The present disclosure relates to single-use lancing devices, for example for use in providing a sample of blood.

### BACKGROUND

Lancing devices are used to obtain blood samples from a person by piercing the skin on a finger or other body part with a small needle or blade. Such blood samples are required for many medical tests, for example glucose tests for diabetes management. To make the process of drawing the blood sample convenient and safe to the user, lancing devices need to satisfy high standards. For example, in order to avoid infection and contamination, lancing devices are preferable single-use disposable products. They must therefore be small and simple, to be economic, and preferably should use a sustainable design to minimise waste and environmental impact through their life cycle.

Disposable lancing devices typically include a needle connected to a holder, collectively referred to as a lancet, and a trigger mechanism. When the trigger is activated the lancet is propelled from a retracted position within the housing of the device to an extended position wherein the needle projects from the housing to prick the subject's skin. Usually the lancet is driven by a metal spring which is cocked during the assembly of the device and released by the trigger. A problem with these devices is that the use of metal springs contributes to the device's environmental impact during disposal. Furthermore, metal springs require additional space within the device, increasing the amount of plastic required and the size of the packaging. Another problem with current designs is that storing a lancing device with a cocked spring can cause parts to become misshapen through tension, impairing its operation.

### SUMMARY

The present invention has been devised to overcome the problems described above; in particular, to address the need for a lancing device which is more sustainable than current designs.

According to a first aspect of the present invention there is provided a lancing device comprising a housing and a lancet. The lancet comprises a needle and is configured to be movable rearwardly within the housing from an initial operational position to a retracted position. The lancing device further comprises a shroud coupled movably to the housing and at least partially covering and extending forwards beyond a forward end of the needle before use of the lancing device, and a biasing member coupled between the lancet and the shroud. The shroud is configured to, when pressed against skin around a sampling site, move rearwards relative to the housing, prime the biasing member during the rearward movement, and, at a first point of the rearward movement, allow the needle to contact the sampling site and subsequently, at a second point of the rearward movement, release energy stored in the biasing member to drive the lancet from the operational position to the retracted position. The housing comprises one or more deflectable legs. The or each deflectable legs extend from an inner surface of the housing and are configured to engage with a shoulder of the lancet to restrict a rearward movement of the lancet with respect to the housing until the shroud reaches the second point of the rearward movement. The shroud is further configured to, at the second point of the rearward movement, deflect the legs so that rearward movement of the lancet is no longer restricted and energy stored in the biasing member is released to drive the lancet from the operational position to the retracted position.

The biasing member may comprise one or more springs that are compressed during the rearward movement. The or each spring may be a plastic spring. The or each plastic spring may be formed integrally with the shroud. The or each plastic spring may be a flexible leg.

The housing may comprise one or more fingers configured to resist an initial rearward movement of the shroud until a level of force applied by the user exceeds a predefined threshold force. The housing and the shroud may comprise cooperating snap-fit fastening features for preventing resetting of the shroud, after the shroud reaches the second point of the rearward movement.

The housing may comprise a lower housing part and an upper housing part in snap-fit engagement with one another. The lower housing part may be configured to couple the shroud moveably to the lower housing. The lower housing part may comprise the or each deflectable legs, one or more fingers configured to resist an initial rearward movement of the shroud until a level of force applied by the user exceeds a predefined threshold force, and snap-fit fastening features for cooperatively engaging with features of the shroud for preventing resetting of the shroud, after the shroud reaches the second point of the rearward movement.

Components of the device may be configured to facilitate assembly of the device by installing the shroud into the lower housing part by inserting the shroud from a lower end of the lower housing part and pushing the shroud rearwards into the lower housing part until the or each finger restricts further rearward movement of the shroud; installing the lancet into the lower housing part by inserting the lancet from an upper end of the lower housing part and fastening the lancet into the or each deflectable legs, and joining the upper housing part with the lower housing part by snap-fit engagement.

### BRIEF DESCRIPTION OF THE DRAWINGS

FIG. 1 shows a side, a front and a horizontal cross-section view of an assembled lancing device;

FIG. 2 is an exploded view of the lancing device of FIG. 1;

FIG. 3 shows cross-sectional side and front views of the lancing device of FIG. 1;

FIG. 4 is a view of a lancet of the lancing device of FIG. 1 after the needle cap has been removed from the lancet;

FIG. 5 shows cross-sectional side and front views of the lancing device of FIG. 1 after removal of the needle cap;

FIG. 6 shows cross-sectional side and front views of the lancing device of FIG. 1 in use where a needle is inserted into the skin;

FIG. 7 shows cross-sectional side and front views of the lancing device of FIG. 1 in use where the needle is further inserted into the skin;

FIG. 8 shows cross-sectional side and front views of the lancing device of FIG. 1 in use where the needle reached a predetermined skin penetration depth;

FIG. 9 shows cross-sectional side and front views of the lancing device of FIG. 1 after usage; and

FIG. 10 shows a cross-sectional side of an alternative embodiment including a compression spring.

### DETAILED DESCRIPTION

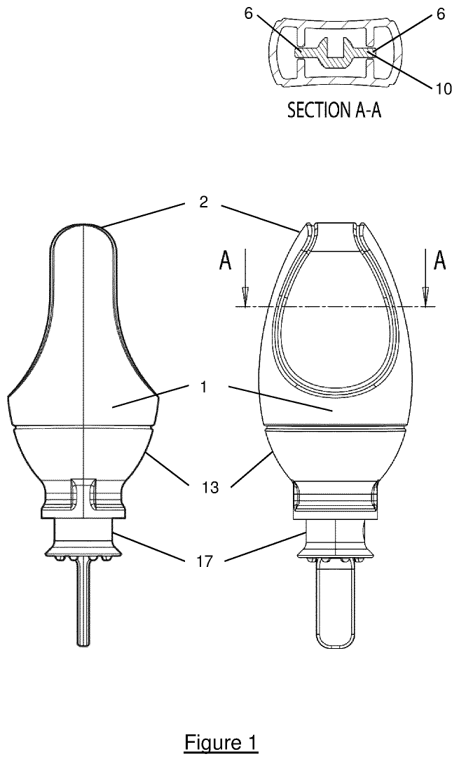

The Figures illustrate the components and operation of a single use lancing device designed for pricking a person's skin to, for example, provide a blood sample. The references to “lower”, “upper”, “horizontal” and “vertical” have been chosen for convenience, and refer to the orientations as shown in FIG. 1 and FIGS. 4-9.

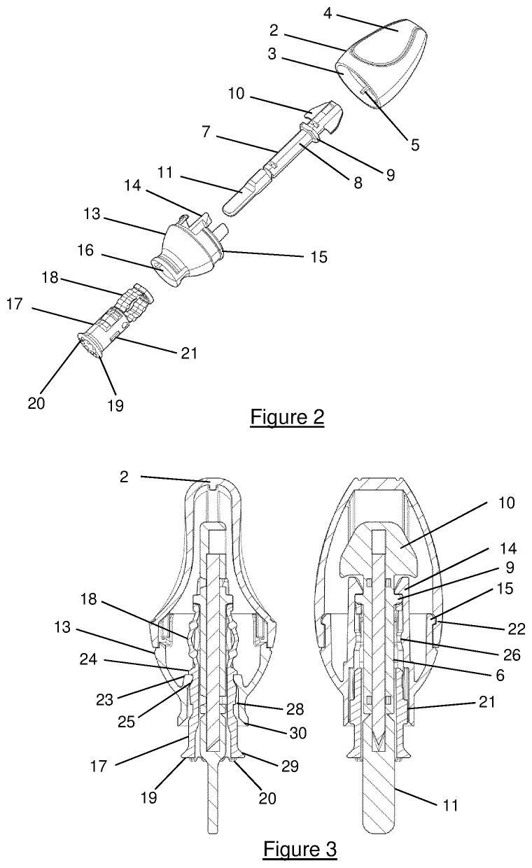

Referring to FIG. 1, a lancing device 1 is shown in an assembled state. As further illustrated in the exploded view of FIG. 2, the lancing device 1 has a lower housing part 13, an upper housing part 2, a lancet 7 and a shroud 17. The shroud 17 comprises a generally tubular shaped section and two spring legs 18 extending away from the tubular section in a substantially coaxial direction. The spring legs 18 serve as respective compression springs. In a relaxed state, both spring legs 18 include a central portion of convex shape with respect to the central axis. The lower end of the shroud 17 has a generally ring-shaped skin contacting surface 19. Projections 20 on the skin contacting surface are intended to “confuse” the nerves in the area of the prick to make the prick less painful to the user. The shroud 17 comprises two ribs 21 which extend laterally from an outer surface of the shroud 17.

Referring to FIG. 3, the shroud 17 comprises two hooks 24 which extend laterally from the outer surface of the shroud 17, and which provide generally flat and horizontal surfaces at their lower ends. The outer surface of the shroud 17 provides two tapered portions 25 located below the hooks 24. The tapered portions 25 are narrower at their upper ends than at their lower ends. The outer surface of the shroud 17 provides two laterally extending hooks 28 located below the tapered portions 25, providing generally flat and horizontal surfaces at their lower ends. At its lower end, the shroud 17 provides a tapered lip 29 on the outer surface. The lateral extent of the tapered lip 29 is greater at its lower end than at its upper end.

Referring to FIG. 2, the lower housing part 13 is generally tubular shaped and provides an axial passage 16. The lateral extent of the lower housing part 13 is greater at its upper end than at its lower end. A plug 15 extends from an outer surface of the lower housing part 13. The plug 15 is generally annular shaped. Instead of forming a complete ring, the plug 15 may comprise two portions, which are arc shaped, as illustrated in FIG. 2. As shown in FIG. 3, the lower housing part 13 provides two deflectable fingers 23 which protrude from an inner surface of the lower housing part 13. Each finger 23 comprises an overhanging section which reduces the lateral extent of the passage 16.

The lower housing part 13 also provides two legs 14 which extend upwards from the inner surface of the lower housing part 13 (fewer or more than two legs may also be envisaged). The upper end of each leg 14 is shaped to serve as a hook of a snap-fit fastener. At the upper end of each leg 14, a generally triangular shaped hook protrudes towards the central axis of the lower housing part 13, providing a generally flat and horizontal surface at its lower end.

The legs 14 are deflectable such that the unsupported upper ends of the legs flex away from the central axis of the lower housing part 13 when the legs 14 are deflected. Each of the legs 14 comprises a section 26 which protrudes towards the central axis of the lower housing part 13. As discussed in more detail below, the protruding section 26 is configured to enable an interaction with the shroud 17 such that the shroud 17 deflects the legs 14 during operation of the lancing device. At its lower end, the inner surface of the lower housing part 13 further includes a tapered region 30. As discussed in further detail below, this tapered region 30 is configured to match the tapered lip 29 of the shroud 17.

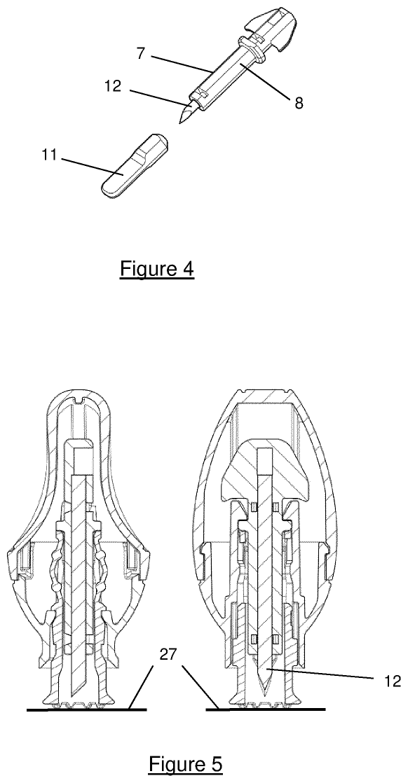

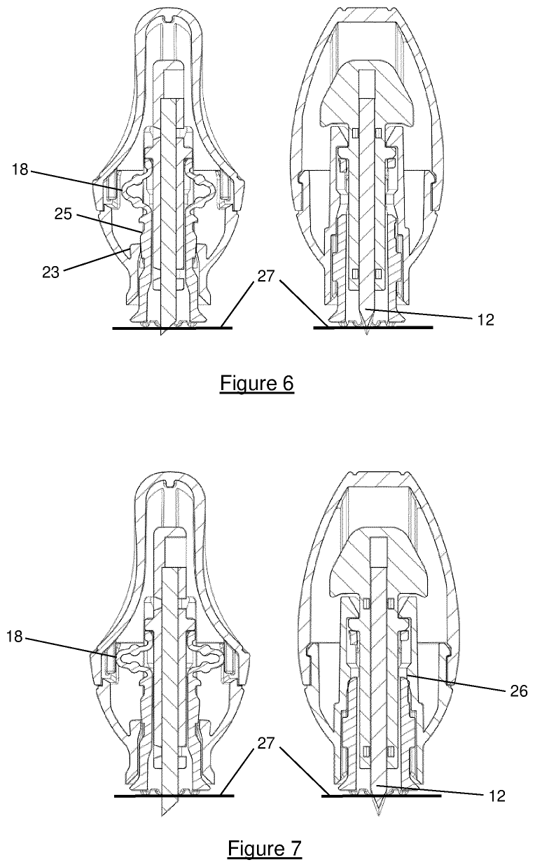

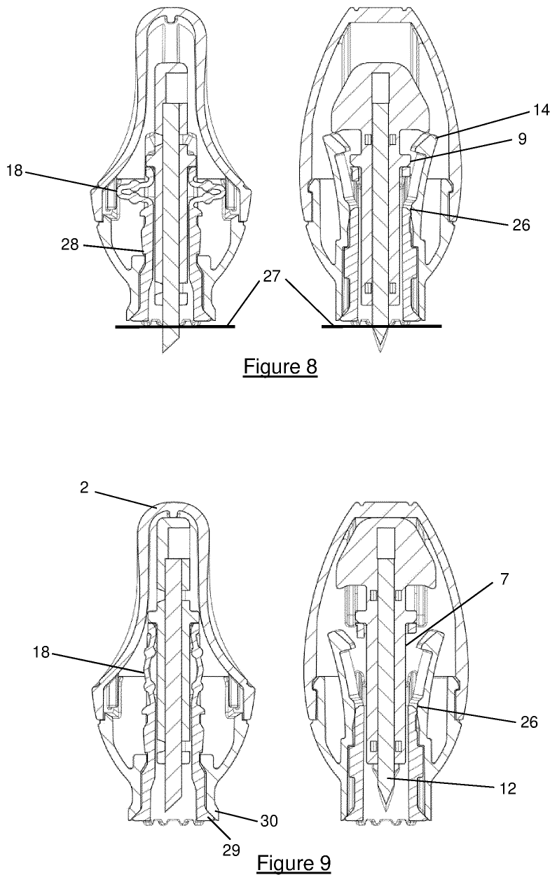

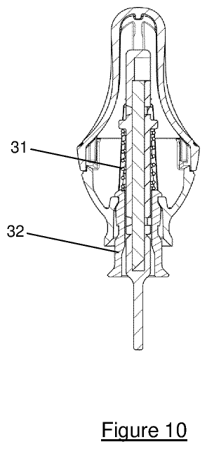

Referring to FIG. 2 again, the lancet 7 comprises an elongated body 8, and a needle 12 embedded within the body 8 and initially shrouded by a cap 11. The body 8 has a laterally extending shoulder 9 which provides generally flat and horizontal surfaces at its lower and upper ends. At an upper end, the body 8 comprises laterally extending ribs 10. The lateral extent of the ribs 10 is greater at its lower end than at its upper end. The cap 11 is moulded integrally with the body 8 but the cap 11 can be twisted off to expose the needle tip extending from a lower end of the body 8. The shape of the cap 11 is sufficiently flat and wide that it can be grasped by a user to facilitate twisting. An initial connection between the cap 11 and the body 8 is sufficiently weak that twisting of the cap 11 relative to the body 8 breaks the cap 11 free without damaging the body 8. FIG. 4 shows the cap 11 and the lancet 7 after the cap 11 has been twisted off.

The upper housing part 2 is closed at its upper end and defines a generally circular opening 3 at its lower end. The lateral extent of the upper housing part 2 is greater at its lower end than at its upper end. The upper housing part 2 includes two portions of convex shape and two portions of concave shape with respect to the central axis, as illustrated respectively in the side and front views shown in FIG. 3. Each concave shaped portion of the upper housing part 2 comprises a grip area 4 to assist a user in grasping the device during operation. FIG. 2 shows only one grip area 4 as the second grip area is located on an opposite side of the upper housing device 2 and is not visible in this view. An inner surface of the opening 2 provides a socket 22 which is generally annular shaped. The socket 22 is configured to be cooperatively engageable with the plug 15 of the lower housing part 13 to join the two housing parts. The upper housing part 2 further comprises a rib 5 which extends downwards and is configured to be engageable with the lower housing part 13 to prevent rotation of the upper and lower housing parts 2, 13 in relation to each other when the two housing parts are joined. As shown in the horizontal cross section in FIG. 1, the upper housing part 2 further defines slots 6 configured to receive the ribs 10 of the lancet 7.

The lower housing part 13, the upper housing part 2, the shroud 17 and the lancet body 8 are moulded components. Considering the lancet, the needle 12 is embedded into the lancet body 8 by over-moulding. It is an advantage that the main components of the lancing device 1, including the spring legs 18, are manufactured from plastic materials. Moulded plastic springs enable designs which are highly compact and reduce waste.

The assembly of the lancing device comprises three simple steps. First, the shroud 17 is pushed into the passage 16 from the lower end of the lower housing part 13 until the fingers 23 restrict further rearward movement of the shroud 17. Second, the lancet 7 is installed in its operating position in the lower housing part 13 by inserting it into the passage 16 from the upper end of the lower housing part 13 and fastening the shoulder 9 into the two legs 14. Third, the upper housing part 2 is connected to the lower housing 13 part via the corresponding annular snap fit features 15, 22. NB. The lancet is sterilised prior to assembly.

In an assembled lancing device, as shown in FIGS. 1 and 3, the lower and upper housing parts 2, 13 are joined together by a snap-fit engagement of the plug 15 and socket 22. The rib 5 of the upper housing part 2 is engaged with the lower housing part 13 to prevent rotation of the upper and lower housing parts 2, 13 in relation to each other. The lancet 7 is retained in an operating position in the lower housing part 13 by the legs 14. An upper end of the shoulder 9 engages with the legs 14, restricting a rearward movement of the lancet 7 with respect to the lower housing part 13. Similarly, a forward movement of the lancet 7 is restricted by the engagement of the legs 14 with the ribs 10 of the body 8. The ribs 10 are received by the slots 6 in the upper housing, preventing axial rotation of the lancet 7. The lower end of the shoulder 9 contacts an upper end of the spring legs 18 of the shroud 17. The spring legs 18 serve as compression springs between the shroud 17 and the shoulder 9. In other words, the retraction spring legs 18 serve as biasing members coupling the lancet 7 and the shroud 17.

In an assembled lancing device, an upper end of the shroud 17 is located inside the lower housing part 13 and the shroud 17 extends outwardly from the passageway 16 of the lower housing part 13. The ribs 21 of the shroud 17 cooperatively engage with pockets in the lower housing part 13, preventing rotational movement of the shroud 17. The hooks 24 of the shroud 17 are cooperatively engaged with recesses in the lower housing part 13, preventing forward movement of the shroud 17. The fingers 23 are engaged with the tapered portions 25 of the shroud 17. The fingers 23 restrict the rearward movement of the shroud 17 until a level of force applied by the user exceeds a predefined threshold force and thereby prevent an accidental activation of the lancing device.

With reference to FIGS. 5 to 9, the operation of the lancing device will now be described. In order to prepare the lancing device 1 for use, a user holds the lancing device with the fingers of one hand by the grip areas 4, grips the cap 11 with the fingers of the other, and twists off the cap 11 to break the connection allowing the cap to be pulled off the needle. As the ribs 10 of the lancet body 8 are located in the slots 6, the lancet 7 is prevented from rotating which allows a user to twist the cap 11 relative to the body 8 to break off the cap 11.

After removal of the cap 11, the user places the skin contacting surface 19 with projections 20 against skin 27 around a sampling site, as illustrated in FIG. 5. At this stage, the shroud 17 extends forwards beyond a forward end of the needle 12, preventing contact of the skin 27 with needle 12. Pressing the lancing device against the skin 27, the user applies a level of force that exceeds the predefined threshold force. Due to the applied force, the tapered portions 25 of the shroud 17 deflect the fingers 23 outwardly, allowing the shroud 17 to move rearwards into the housing, as shown in FIG. 6. Next, the force, which is still applied by the user, leads to a sudden rearward movement of the shroud 17. During the rearward movement of the shroud 17, the lancet remains retained in its operational position by the deflectable legs 14 of the lower housing part 13, and the spring legs 18 of the shroud 17 are compressed along their axes. As shown in FIG. 6, the applied force bends the central portion of each spring leg 18 laterally away from the central axis of the shroud 17. A reliable and predictable deformation of the spring legs 18 upon compression is ensured by the design of the spring legs 18, and in particular by the convex shape of the central portion. Upon reaching a first point of the rearward movement of the shroud 17, the needle 12 contacts the sampling site.

The user then continues to press the lancing device 1 against the skin 27 and, as illustrated in FIG. 7, the shroud 17 moves further rearwards, the needle 12 further penetrates the skin 27, and the spring legs 18 are further compressed. As illustrated in FIG. 8, at a second point of the rearward movement of the shroud 17, the needle 12 reaches a predetermined skin penetration depth. The shroud 17 is now largely retracted into the housing parts 2, 12 while further rearward movement of the shroud 17 is prevented by the lip 29 contacting the tapered region 30 of the lower housing 12. At this stage, the hooks 28 of the shroud 17 engage with the fingers 23 of the lower housing 12, so that forward movement of the shroud 17 is also prevented. The shroud 17 is now essentially locked in place. It is an advantage that the lip 29 has a tapered shape and that the tapered region 30 is configured to match the lip 29 as this restricts access to pull the shroud 17 back out thereby preventing improper resetting of the device.

The legs 14 are now flexed away from the central axis of the lower housing part 13 by the shroud 17 engaging with the protruding section 26 of the legs 14. The upper ends of the legs 14 are no longer engaged with the lancet shoulder 9. The rearward movement of the lancet 7 is no longer restricted within the housing and the energy stored in the spring legs 18 is released to drive the lancet 7 from the operational position to a retracted position. As shown in FIG. 9, when the lancet 7 is in the retracted position, the needle 12 does not extend outwardly from the passageway 16 of the lower housing part 13. Retracting the needle 12 when the moveable shroud 17 reaches a predefined position provides the advantage of a consistent and well-defined skin penetration depth.

Next, the user removes the lancing device from the sampling site and collects the blood sample, e.g. with a suitable swab. As the lancet 7 is now in the retracted position, the risk of accidental needle injuries is greatly reduced. The lancing device is now in a “used” state and can be disposed of. The retracted shroud 17 serves as a visual indicator to the user that the device has been used and thereby prevents any confusion by the user as to the state of the lancing device.

It is an advantage of the lancing device 1 that the shroud 17 engages directly with the legs 14 to release the lancet 7 since this allows precise control over the maximum skin penetration depth without requiring overly tight (and thus expensive) manufacturing tolerances. As described above, the needle 12 penetrates further into the skin until the lancet is retracted, i.e. until the shroud 17 engages with the protruding section 26 of the legs 14 to flex the legs 14 away from the central axis of the housing. This simple release mechanism may be less sensitive to manufacturing variations than known release mechanisms which employ intermediate parts to realise a coupling between the shroud and the lancet retaining features. In particular, the maximum skin penetration depth of the lancing device 1 does not depend on the stiffness of the deflectable legs 14 but only on the dimensions of the protruding section 26 of the legs 14 and the corresponding engaging section of the shroud 17. Thus, the lancing device 1 described with respect to the accompanying figures provides a very simple and elegant solution to the problem of manufacturing disposable lancing devices with a consistent and well-defined skin penetration depth.

Those skilled in the art will appreciate that various modifications may be made to the above described embodiment without departing from the scope of the present invention.

In the embodiment shown in FIGS. 1 to 9, the needle 12 does not extend outwardly from the passageway 16 of the lower housing part 13 when the lancet 7 is in the retracted position. In another embodiment, the needle 12 may extend outwardly from the passageway 16 of the lower housing part 13 when the lancet 7 is in the retracted position. In this case, the forward end of the needle 12 is still covered by the shroud 17 which, even in the retracted position, extends slightly from the housing.

FIG. 10 shows an alternative embodiment in which the biasing member coupled between the lancet 7 and a modified shroud 32 is a metal compression spring 31. The modified shroud 32 differs from the shroud 17 in that the modified shroud 32 does not include the spring legs 18.

In the above description, the pricking member that pierces the skin has been described as a “needle”. Those skilled in the art will appreciate that this term encompasses conventional needles, blades etc.

| Reference | |
|---|---|
| numeral | Feature |
| 1 | Lancing device |
| 2 | Upper housing part |
| 3 | Opening |
| 4 | Grip areas |
| 5 | Rib |
| 6 | Slots |
| 7 | Lancet |
| 8 | Body |
| 9 | Shoulder |
| 10 | Lancet ribs |
| 11 | Needle cap |
| 12 | Needle |
| 13 | Lower housing part |
| 14 | Legs |
| 15 | Plug |
| 16 | Passageway |
| 17 | Shroud |
| 18 | Spring legs |
| 19 | Skin contact area |
| 20 | Projections |
| 21 | Shroud ribs |
| 22 | Socket |
| 23 | Finger |
| 24 | Hooks |
| 25 | Tapered portions |
| 26 | Protruding section |
| 27 | Skin |
| 28 | Lower shroud hooks |
| 29 | Shroud lip |
| 30 | Lower housing tapered region |
| 31 | Compression spring |
| 32 | Modified shroud |

1-9. (canceled)

10. A lancing device comprising:
a housing;
a lancet comprising a needle, the lancet being configured to be movable rearwardly within the housing from an initial operational position to a retracted position;
a shroud coupled movably to the housing and at least partially covering and extending forwards beyond a forward end of the needle before use of the lancing device; and
a biasing member coupled between the lancet and the shroud,
wherein the shroud is configured to, when pressed against skin around a sampling site, move rearwards relative to the housing and, at a first point of the rearward movement, allow the needle to contact the sampling site and subsequently, at a second point of the rearward movement, release energy stored in the biasing member to drive the lancet from the operational position to the retracted position, and
wherein the housing comprises lancet retaining features comprising one or more deflectable legs configured to retain the lancet in the operational position until the shroud reaches the second point of the rearward movement.

11. A lancing device according to claim 10, wherein the shroud is configured to prime the biasing member during the rearward movement.

12. A lancing device according to claim 10, wherein the biasing member comprises one or more springs that are compressed during the rearward movement.

13. A lancing device according to claim 12, wherein the or each spring is a plastic spring.

14. A lancing device according to claim 13, wherein the or each plastic spring is formed integrally with the shroud.

15. A lancing device according to claim 14, wherein the or each plastic spring is a flexible leg.

16. A lancing device according to claim 10, wherein the housing comprises one or more fingers configured to resist an initial rearward movement of the shroud until a level of force applied by the user exceeds a predefined threshold force.

17. A lancing device according to claim 10, wherein the housing and the shroud comprise cooperating snap-fit fastening features for preventing resetting of the shroud, after the shroud reaches the second point of the rearward movement.

18. A lancing device according to claim 10, wherein the housing comprises a lower housing part and an upper housing part in snap-fit engagement with one another, said lower housing part is configured to couple the shroud moveably to the lower housing, and said lower housing part comprises:
the lancet retaining features;
one or more fingers configured to resist an initial rearward movement of the shroud until a level of force applied by the user exceeds a predefined threshold force, and
snap-fit fastening features for cooperatively engaging with features of the shroud for preventing resetting of the shroud, after the shroud reaches the second point of the rearward movement.

19. A lancing device according to claim 18, wherein components of the device are configured to facilitate assembly of the device by:
installing the shroud into the lower housing part by inserting the shroud from a lower end of the lower housing part and pushing the shroud rearwards into the lower housing part until the or each finger restricts further rearward movement of the shroud;
installing the lancet into the lower housing part by inserting the lancet from an upper end of the lower housing part and fastening the lancet into the lancet retaining features, and
joining the upper housing part with the lower housing part by snap-fit engagement.

20. A lancing device according to claim 10, wherein the one or each deflectable leg extends from an inner surface of the housing and is configured to engage with a shoulder of the lancet to restrict a rearward movement of the lancet with respect to the housing until the shroud reaches the second point of the rearward movement.

21. A lancing device according to claim 10, wherein the shroud is further configured to, at the second point of the rearward movement, deflect the or each leg so that rearward movement of the lancet is no longer restricted and energy stored in the biasing member is released to drive the lancet from the operational position to the retracted position.
